(19) United States
(12) Patent Application Publication
(10) Pub. No.: US 2008/0132895 A1
(43) Pub. Date: Jun. 5, 2008

(54) Instruments and method for arthroscopic arthroplasty of the knee

(76) Inventor: Ron Clark, Valparaiso, IN (US)

Correspondence Address: Mark S. Morrison, 1508 Springhill Rd., Warsaw, IN, 46580, US

(21) Appl. No.: 11/197,060

(22) Filed: Aug. 5, 2005

(51) Int. Cl. A61B 17/58

(52) U.S. Cl. 606/79; 606/102; 606/88; 606/103

(57) ABSTRACT

Instruments and method for the arthroscopy arthroplasty of a knee joint, including instruments and method for the use of the instruments in the resection of tibial and femoral surfaces and the implantation of tibial and femoral components. In resecting the femur, a femoral cutting mill is attached to the tibial surface with the knee in a flexed position. With the cutting mill activated, the knee is extended causing the cutting surface of the femoral cutting mill to engage the femoral surface and form a trough in the femoral surface for receipt of the femoral component. A network of channels formed within the prosthetic components is used for the delivery of bone cement to the component bone interface.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO MICROFICHE APPENDIX

Not Applicable.

### BACKGROUND OF THE INVENTION

The present disclosure relates generally to instruments and methods for use in knee arthroplasty, and more particularly, but not necessarily limited to, methods for arthroscopic arthroplasty of the knee.

It is common to provide implants to resurface worn articular surfaces of knees. Many of the prior art surgical instruments and procedures require large incisions to gain adequate access to the joint space to perform the surgery and the removal of a great deal of bone from the femur and tibia in order to accommodate the implant, thereby causing large amounts of surgical trauma to the patients and reducing the amount of bone available in the event that revision surgery is required. In addition, the removal of too much bone may lead to failure of the implanted prosthesis due to subsidence of the implant into the underlying bone necessitating revision surgery to replace the failed implants.

Bone cement is typically used to secure implant components. Misalignment of components may occur when bone cement is placed on the prepared bone surface before the components are implanted into position. When a component is implanted into bone cement which has been placed on the prepared bone surface, bone cement may escape from between the bone and the edges of the implant. When bone cement is injected into a portal located on a side wall of an implant, bone cement may leak from the portal into the joint space. If left in the joint space, such excess or leaked bone cement may cause irritation. On the other hand, inadequate amounts of bone cement may result in inadequate fixation resulting in the loosening of the implant.

### BRIEF SUMMARY OF THE INVENTION

Accordingly, it is a primary object of the present invention to overcome the above mentioned disadvantages associated with prior art instruments and surgical procedures.

Another object of the present invention is to reduce surgical trauma by providing surgical instruments and procedures that reduce the required size of surgical incisions and that reduce the amount of bone that must be removed during surgery when compared to prior art surgical instruments and procedures.

Another object of the present invention is to reduce the leakage of bone cement into the joint space by providing an improved system for delivering bone cement to the interface between the implant and the bone.

Another object of the present invention is to reduce the likelihood of subsidence of implants by providing a surgical procedure that requires a minimal resection of bone such that the bone architecture is left intact and better able to adequately support implanted devices.

Another object of the present invention is to provide a surgical procedure that, in the event that revision surgery is required, would allow the use of a standard unicompartment replacement knee prosthesis in the revision surgery by providing a surgical procedure that requires a minimal resection of bone such that the bone architecture is left intact leaving more bone stock available for use in revision surgery when compared to prior art surgical procedures.

Instruments and method for the arthroscopy arthroplasty of a knee joint are disclosed, including instruments and methods for the use of the instruments in the resection of tibial and femoral surfaces and the implantation of tibial and femoral components. In resecting the femur, a femoral cutting mill is attached to the tibial surface with the knee in a flexed position. With the cutting mill activated, the knee is extended causing the cutting surface of the femoral cutting mill to engage the femoral surface and form a trough in the femoral surface for receipt of the femoral component. The use of a network of channels which are formed within the prosthetic components for the delivery of bone cement is disclosed.

### BRIEF DESCRIPTION OF THE DRAWINGS

The features and advantages of the disclosure will become apparent from a consideration of the subsequent detailed description presented in connection with the accompanying drawings in which:

FIG. 1 is a top view of a tibial cutting guide in accordance with the principles of the present invention; and

FIG. 2 is a perspective view of a trial femoral prosthesis and template in accordance with the principles of the present invention.

### DETAILED DESCRIPTION OF THE INVENTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the invention claimed.

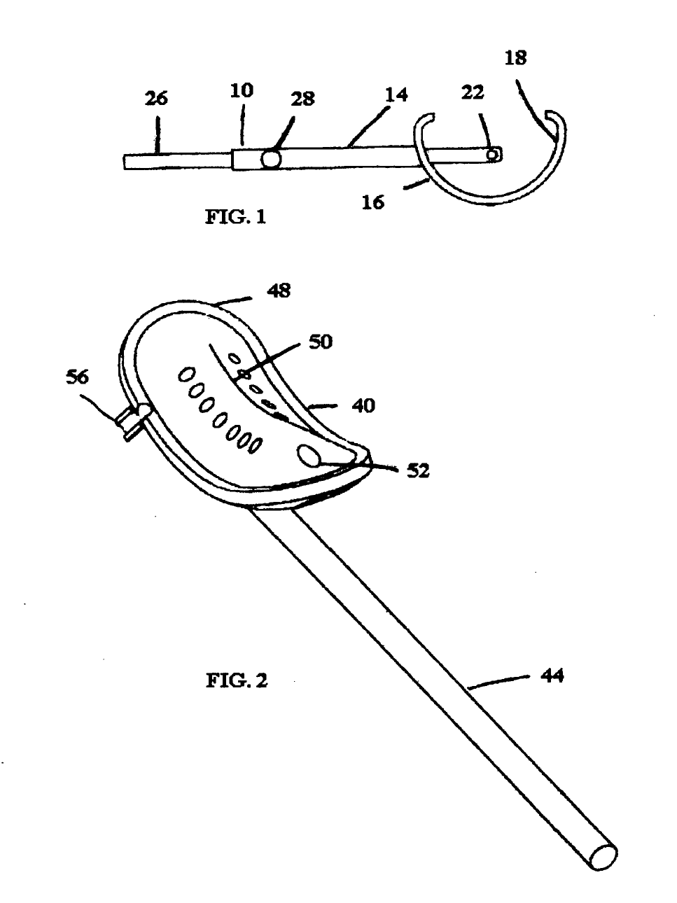

The tibial cutting guide 10 in accordance with the present invention is illustrated in FIG. 1. The tibial cutting guide is used to guide a cutting tool, mill or burr in removing about one half or more of the material that needs to be removed to prepare the tibial surface to receive a tibial implant. The cutting guide includes an elongated handle 14 with a cutting guide arm 16 mounted on the distal end of the handle. The inner surface 18 of the cutting guide arm is in the size and shape of one half or more of the periphery of the tibial implant and will be used to guide the cutting tool in removing material from the tibial surface. A retractable foot 22 may be mounted on the end of a rod 26 moveably mounted within a bore through the handle 10. A knob 28 on the handle is used to tighten a set screw in the handle which can be tightened to lock the rod 26 in place or loosened to permit the rod to be moved.

The trial femoral prosthesis and template 40 in accordance with the present invention is illustrated in FIG. 2. The trial femoral prosthesis and template includes an elongate handle 44 with a trial prosthesis body 48 mounted on the distal end. The proximal surface 50 of the trial prosthesis matches the size and shape of the femoral implant that is to be implanted. The trial femoral prosthesis and template may have a notch 56 on its periphery. The notch may be used to guide the placement of a guide wire into the femur. A bore 52 through the trial prosthesis body may be used to guide a drill in preparing a hole for a bone screw or other fastener.

The method of arthroscopic arthroplasty of the knee in accordance with the invention is hereinafter described.

A compartment of human knee is selected for replacement of bearing surfaces with prosthetic devices.

An arthroscope is placed in a portal opposite the compartment to be resurfaced. A standard arthroscope is utilized. After initial arthroscopy with debridement and the removal of synovial debris and meniscal rim as may be necessary to assure adequate visualization of the joint space in the compartment of the knee being resurfaced, the arthroscope is used to visualize the preparation of the bearing surfaces for receiving implants and the implantation of prosthetic devices within the joint.

An ipsilateral portal is formed through the soft tissue of the knee to provide instrument access to the joint space within the compartment to be resurfaced. This ipsilateral portal may be from about 5 mm to about 75 mm in length, typically about 5 mm to about 25 mm in length, and preferably about 10 mm in length. Additional accessory portals may be made as necessary.

A calibrated probe comprising a shaft with a hook on the distal end is inserted through the ipsilateral portal and hooked to the posterior edge of the tibial surface. Markings on the shaft of the probe are used to determine the anterior to posterior dimension of the tibial surface. The anterior to posterior dimension of the tibial surface is used by the surgeon to select the appropriate size of the tibial implant to be implanted.

The medial to lateral dimension is determined by using a rotating arm located on the shaft of the probe which can be interchangeably moved along the shaft to positions that correspond to different implant sizes. The surgeon may use a second arthroscopic probe to rotate the arm into a medial or lateral position to locate the center point of the tibial surface to be resected. The center point of the tibial surface is marked by driving a pointed awl downward into the center point of the tibial or, alternatively, by forcing a flexible pointed wire that traverses the internal axis of the calibrated probe and exits inferiorly at the predetermined center point. The center point mark is enlarged to a 3 to 4 mm diameter center point defect in the tibial surface using a rotating burr so as to clearly define the location of the center point of the tibial surface to be resurfaced.

With the center point of the tibia located and marked and with the appropriate size of the tibial implant selected, a tibial cutting guide that corresponds to the selected tibial implant is inserted through the ipsilateral portal using a handle attached to the tibial cutting guide.

The guide may have a retractable foot that can be extended to sit in the center point defect. After the retractable foot is placed in the center point defect, the alignment of the tibial cutting guide may be adjusted by medial lateral movement of the handle. Thus positioned, the cutting guide surface of the tibial cutting guide will be accurately located to guide the resection of the tibial surface to receive a tibial implant.

To provide additional stability to the tibial cutting guide when in use, additional means to secure the guide to the tibia, such as an eyelet that can be traversed by a wire drilled into the tibia, or such as a passageway in the shaft of the tibial cutting guide for passing a flexible drill wire so that the wire could be inserted through an external orifice in the shaft and then passed into the joint and then into the bone of the tibia, may be provided.

After the tibial surface cutting guide is properly aligned upon the surface of the tibia, a tibial cutting mill or burr is introduced into the joint space through an accessory portal located about 10 mm superior to the ipsilateral portal. Alternatively, if the ipsilateral portal is large enough, the tibial cutting mill may be introduced through the same portal. The tibial cutting mill or burr, guided by guide surfaces on the tibial surface cutting guide, is used to remove material from the proximal surface of the tibia. In the preferred embodiment, a depth of about two to four mm of the surface material is removed during the surface removal process.

Following use of the tibial cutting mill or burr, the fastener system securing the tibial surface cutting guide to the surface of the tibia, if used, should be removed and the tibial surface cutting guide should be removed from the joint space through the portal.

The tibial surface cutting guide as shown in FIG. 1 prepares about one half the surface area of the tibia required for the implantation of a tibial prosthetic implant. The second tibial hemisurface is prepared in the same manner as the first. A tibial surface guide that has a cutting guide arm that defines the shape of the second hemisurface is used. If the tibial implant is bilaterally symmetrical, the tibial surface cutting guide that was used to prepare the first hemisurface may be turned over and used to prepare the second hemisurface. If the tibial implant is not bilaterally symmetrical, then the first tibial surface cutting guide, whether it was configured and used to remove material from the medial or lateral hemisurface, should be removed from the joint space and replaced with a second tibial surface cutting guide of the appropriate size which is configured to be used to remove material from the second hemisurface. The tibial surface cutting guide may be aligned and secured as for the preparation of the first hemisurface. The second tibial hemisurface is prepared in the same manner as the first. Following use of the tibial cutting mill or burr to remove material from the second tibial hemisurface, the fastener system securing the tibial surface cutting guide to the surface of the tibia, if used, should be removed. The tibial cutting guide may be removed from the joint space.

A trial tibial prosthesis may be placed through an ipsilateral portal and into the defect cut into the tibia. Any modifications to the tibia may be made as needed. Preparation of the tibial surface to receive a tibia implant is complete.

With the tibial surface prepared, attention is then directed to the femoral surface. A calibrated awl that is curved to fit the distal femoral articulating surface in dimensions to corresponding femoral implant options is inserted into the joint space through an ipsilateral portal. By viewing arthroscopically and flexing and extending the joint, the surgeon will be able to ascertain the correct size of femoral implant to be selected. Once the appropriate sized awl is determined and the location of implantation selected, the awl is driven into the femur with a mallet to inscribe the alignment and endpoints of the femoral implant into the surface of the femur and the awl is withdrawn from the joint space.

A femoral cutting mill is passed into the joint space through one of the portals using a handle attached to the femoral cutting tool. The knee joint is flexed to facilitate the placement of the cutting tool into position upon the surface of the tibia before being removeably secured to the tibia. The surface of the femoral cutting mill that faces the tibia has a base that may be configured to fit within the prepared surface of the tibia. When the base is inserted into the prepared surface of the tibia, the cutting tool is properly in position and alignment with the femur to resect the appropriate amount of bone from the femur. The surface of the cutting tool which faces the femur bears a sharpened tool bit such as a burr that will be used to cut the surface of the femur while the undersurface of the cutting tool may have a post or a sharpened point to be driven into the location of the center point defect that had been formed on the tibial surface to stabilize the cutting tool onto the tibia. The undersurface of the cutting mill may have one or more threaded fasteners that may be turned into the tibial surface to removeably secure the cutting tool to the tibial surface.

With the knee in a flexed position and with the femoral cutting mill positioned on the tibial surface, the femoral cutting mill is activated and the knee is slowly extended causing the cutting surface of the femoral cutting mill to engage the femoral surface and form a trough in the femoral surface which will correspond with the shape of the proximal surface of the femoral prosthetic implant. The marks which were placed onto the femoral surface are used to assist the surgeon in proper placement and length of the trough. Following the formation of the trough, the femoral cutting mill is removed.

A trial femoral prosthesis and template is then placed through an ipsilateral portal and into the defect cut into the femur. Any modifications to the femoral cut can be made as needed.

When a flanged femoral prosthesis is to be implanted, an additional step is needed. With the trial prosthesis in position in the defect cut into the femur, a notch located along the lateral surface of the trial prosthesis in the medial compartment (or located medially along the surface of a trial prosthesis in the lateral compartment) is used to direct a guide wire into the femur from an accessory portal located opposite the trial prosthesis and template. With the wire in position, the femoral trial is then removed. A cannulated, round end cutting mill is then passed over the wire and used to inscribe a circular defect into the femur approximately 4 mm deep. This defect will meet the edge of the femoral implant margin at its mid-circumference and will correspond to a flange that may be on the internal edge of the implant posteriorly (laterally on a medial implant and medially on a lateral implant).

A trial femoral prosthesis and template is then placed through an ipsilateral portal and into the defect cut into the femur. Any modifications to the femoral cut can be made as needed.

Provisional components may be inserted through the superior portal and used to insure proper positioning, range of motion, and knee stability prior to permanent implantation.

In the first embodiment of the method of arthroplasty of the knee, the tibial defect has bone cement placed into it by a delivery system. Bone cement may be delivered into the prepared tibial surface by placing a cement delivery tube through an arthroscopic portal and pumping the cement into the prepared tibial surface. The tibial prosthesis is passed into the knee through a portal and seated into the prepared bed of bone cement on the tibia. Extraneous cement is removed after compressing the implant into the bed.

Bone cement may be applied to the femoral implant as well as be delivered into the exposed femoral defect in a manner similar to the methods used in delivering bone cement into the prepared tibial surface. The femoral implant is passed through a portal and seated into the prepared bed of bone cement on the femur. A screw is placed through the femoral implant anteriorly and, if the a flanged femoral prosthesis is to be implanted, a screw is placed through the flange.

Extraneous bone cement found in the joint space is removed.

Final irrigation of the joint can then be made and assessment of the implantation visualized along with range of motion performed.

Bone cement may be placed into the prepared tibial surface either by placing a cement delivery tube through the bore from the anterior external tibial cortex through the central aspect of the surface of the tibial plateau and pumping the cement into the prepared tibial surface, or by placing a cement delivery tube placed through an arthroscopic portal and pumping the cement into the prepared tibial surface.

The tibial implant may then be placed through an arthroscopic portal and directed into the prepared tibial surface. The tibial implant may then be secured to the tibia by a threaded bolt or other fastener placed through the bore from the anterior external tibial cortex.

In a second and preferred embodiment, the method of arthroplasty of a knee is to be used with implants that have a body with a bore on an external surface within the joint space that is in fluid communication with a network of channels formed in the body for delivery of bone cement through openings on the outer surface of the body into the interface between the body of the implant and the prepared bone surface. The method used in this preferred embodiment is the same as the method used in the first embodiment described above except for the way in which the bone cement is delivered.

In the second embodiment, the preparation of the tibial and femoral surfaces to receive the implants is the same as described in the first embodiment. After the tibial and femoral surfaces are prepared to receive implants, the tibial implant is introduced into the joint space through a portal and placed in the prepared tibial surface before bone cement is introduced in the joint space.

A tube which is attached to a high pressure bone cement delivery system is introduced through a portal into the joint space and placed within a bore on the external, proximal surface of the tibial implant. Bone cement is pumped through the tube into the bore and through the network of channels into the interface between the body of the implant and the prepared tibial surface. The bone cement delivery tube is then removed. The bore on the external surface of the tibial implant may be capped with a cap made of biocompatible material or may be filled with a bone screw. Additional screws may be used to secure the tibial implant to the tibia by inserting screws through any screw holes which may be provided in the tibial implant.

The femoral implant is introduced into the joint space through a portal and placed in the prepared femoral surface before bone cement is introduced in the joint space.

A tube which is attached to a high pressure bone cement delivery system is introduced through a portal into the joint space and placed within a bore on the external, distal surface of the femoral implant. Bone cement is pumped through the tube into the bore and through the network of channels into the interface between the body of the implant and the prepared femoral surface. The bone cement delivery tube is then removed. The bore on the external surface of the femoral implant may be capped with a cap made of biocompatible material or may be filled with a bone screw. Additional screws may be used to secure the femoral implant to the femur by inserting screws through any screw holes which may be provided in the femoral implant.

Extraneous bone cement found in the joint space may be removed.

Final irrigation of the joint can then be made and assessment of the implantation visualized along with range of motion performed.

Routine wound closure and dressings are then applied.

In a third embodiment, the method of arthroplasty of the knee is to be used with implants that have a body with a bore on an external surface that is within interface between the implant and the prepared bone surface. The bore on the external surface is in fluid communication with a network of channels formed in the body for delivery of bone cement through openings on the outer surface of the body into the interface between the body of the implant and the prepared bone surface. The method used in this third embodiment is the same as the method used in the second embodiment except for the way in which the bone cement is delivered.

In the third embodiment, the preparation of the tibial and femoral surfaces to receive the implants is as described in the second embodiment. In addition, as described below, a bore is formed in each bone that is to receive an implant as described below.

After the tibial and femoral surfaces are prepared to receive implants, the tibial implant is introduced into the joint space through a portal and placed on the prepared tibial surface before bone cement is introduced in the joint space.

The tibial implant is secured to the prepared surface of the tibia with a cannulated fastener placed through a drill hole which was formed through the tibia from an external surface of the tibia into the prepared surface of the tibia. The cannulated fastener is secured within a transverse bore formed on the distal, external surface of the tibial implant. The bore may be internally threaded or otherwise adapted to receive and be secured to the fastener. The bore to which the cannulated fastener is fastened is in fluid communication with a network of channels formed in the body for delivery of bone cement through openings on the outer surface of the body into the interface between the body of the implant and the prepared surface of the tibia. A high pressure cement delivery system is connected to a cannulated fastener and is used to pump bone cement through the fastener into the bore and into the network of channels and thus into the interface between the tibial implant and the prepared surface of the tibia. Following delivery of the bone cement, the cement delivery system is disconnected from the fastener. Additional screws may be used to secure the implant to the bone. Extraneous cement, if any, which leaks from the periphery of the tibial implant may removed.

The femoral implant is secured with a cannulated fastener placed through a drill hole which was formed through the femur substantially perpendicular to the long axis of the femoral implant and substantially parallel with the axis of rotation of the knee and into the prepared surface of the femur. The cannulated fastener is secured within a transverse bore formed on the proximal, external surface of the femoral implant. The bore may be internally threaded or otherwise adapted to receive and be secured to a fastener. The bore to which the cannulated fastener is fastened is in fluid communication with a network of channels formed in the body for delivery of bone cement through openings on the outer surface of the body into the interface between the body of the implant and the prepared surface of the femur. A high pressure cement delivery system is connected to the cannulated fastener and is used to pump bone cement through the fastener into the bore and into the network of channels and thus into the interface between the femoral implant and the prepared femoral surface. Following delivery of the bone cement, the cement delivery system is disconnected from the fastener. Additional screws may be used to secure the implant to the bone. Extraneous cement, if any, which leaks from the periphery of the femoral implant may removed under direct arthroscopic visualization.

Final irrigation of the joint can then be made and assessment of the implantation visualized along with range of motion performed.

Routine wound closure and dressings are then applied.

The method of arthroscopic arthroplasty of the knee of this invention is suitable for use in either compartment of the knee. An arthroscopic total knee arthroplasty could be achieved by using the method of this invention in both compartments of the knee.

What is claimed is:

1. An probe for use in the arthroplasty of the tibial surface of the knee comprising a shaft that has a hook on the distal end and that has calibrated markings along the shaft for use in determining the anterior to posterior dimension of the tibial surface.

2. The probe of claim 1 wherein the shaft of the probe is also provided with a rotating arm which may be moved along the shaft to preselected positions that correspond to different sizes of tibial implants and which may be rotated into a medial or lateral position and be used to locate the center point of the area of the tibial surface to be resected.

3. The probe of claim 1 wherein the shaft of the probe is hollow and may conduct a flexible wire or other device that will exit the probe inferiorly at a predetermined location.

4. A tibial cutting guide comprising an elongated handle with a cutting guide arm located on the distal end wherein the cutting guide arm is in the size and shape of about one half or more of the area of the tibial surface which is to be resected.

5. The tibial cutting guide of claim 4 wherein a retractable foot is mounted on the end of a rod moveably mounted within a bore through the handle.

6. A femoral bone resecting tool comprising a proximal portion that rotates an instrument against the femoral surface and a distal surface that rests against the tibia.

7. The femoral bone resecting tool of claim 6 wherein the tool has a sharpened point or threaded mechanism on its distal surface that may provide stability by penetrating the tibial surface.

8. A trial femoral prosthesis and template comprising an elongate handle with a trial prosthesis body mounted on the distal end wherein the trial prosthesis body has a notch on its periphery for use in guiding the placement of a guide wire.

9. The trial femoral prosthesis and template of claim 8 wherein the trial prosthesis body has a through bore for use as a drill guide.

10. A method of arthroscopic knee arthroplasty comprising the steps of
making a portal through the soft tissue of the compartment of the knee opposite the compartment to be resurfaced;
inserting an arthroscope into the portal and visualizing the joint space in the compartment of the knee to be resurfaced;
making an ipsilateral portal through the soft tissue of the knee for instrument access to the compartment to be resurfaced;
locating the area of the surface of the tibia to be resected to receive a tibial implant;
resecting the area of the tibial surface to be resected using a tibial cutting guide inserted through an ipsilateral portal to guide a cutting tool which is also inserted through a portal;
locating the area of the femoral surface to be resected to receive a femoral implant;
inserting a femoral cutting mill into the joint space through a portal and securing the mill to the tibial surface;
resecting the femoral surface by extending the knee from a flexed position while the femoral cutting mill is activated;
inserting a tibial implant through a portal and securing it to the prepared tibial surface; and
inserting a femoral implant through a portal and securing it to the prepared tibial surface.

11. The method of claim 10 wherein the the tibial implant is secured to the tibial surface using bone cement.

12. The method of claim 11 wherein the tibial implant is additionally secured to the tibial surface using bone screws.

13. The method of claim 11 wherein the bone cement is placed in the prepared tibial surface before the tibial insert is placed onto the prepared tibial surface.

14. The method of claim 11 wherein the bone cement is delivered to the interface between the distal surface of the implant and the prepared surface of the tibia through a network of channels with the body of the implant which have openings on the distal surface of the implant by injecting the bone cement under pressure through a bore on the external surface of the implant which is in fluid communication with the network of channels.

15. The method of claim 14 wherein the bore on the external surface of the tibial implant is on the proximal surface of the tibial implant.

16. The method of claim 14 wherein the bore on the external surface of the tibial implant is on the distal surface of the implant.

17. The method of claim 16 wherein the bone cement is delivered to the bore on the external surface of the bibial implant through a cannulated crosspin that is left in place to provide additional fixation to the construct.

18. The method of claim 16 wherein the boned cement is delivered to the bore on the external surface of the tibial implant through a cannulated crosspin that is removed following injection of the bone cement.

19. The method of claim 10 wherein the the femoral implant is secured to the femoral surface using bone cement.

20. The method of claim 19 wherein the femoral implant is additionally secured to the femoral surface using bone screws.

21. The method of claim 19 wherein the bone cement is placed in the prepared femoral surface before the femoral insert is placed onto the prepared femoral surface.

22. The method of claim 19 wherein the bone cement is delivered to the interface between the proximal surface of the femoral implant and the prepared surface of the femur through a network of channels with the body of the implant which have openings on the proximal surface of the implant by injecting the bone cement under pressure through a bore on the external surface of the implant which is in fluid communication with the network of channels.

23. The method of claim 22 wherein the bore on the external surface of the femoral implant is on the distal surface of the femoral implant.

24. The method of claim 22 wherein the bore on the external surface of the femoral implant is on the proximal surface of the femoral implant.

25. The method of claim 24 wherein the bone cement is delivered to the bore on the external surface of the femoral implant through a cannulated crosspin that is left in place to provide additional fixation to the construct.

26. The method of claim 24 wherein the bone cement is delivered to the bore on the external surface of the femoral implant through a cannulated crosspin that is removed following injection of the cement.
